(19) United States
(12) Patent Application Publication
(10) Pub. No.: US 2009/0010993 A1
(43) Pub. Date: Jan. 8, 2009

(54) Composition comprising polyphenol

(75) Inventors: Richard Draijer, Vlaardingen (NL); Ferdinand Alexander Van Dorsten, Vlaardingen (NL); Jozef Gabriel Rita De Mey, Maastricht (NL)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc. d/b/a Unilever

(21) Appl. No.: 12/217,274

(22) Filed: Jul. 2, 2008

(30) Foreign Application Priority Data
Jul. 5, 2007 (EP) EP07111781

(51) Int. Cl. A61K 9/00; A23L 1/29; A61P 9/00; A23L 2/52

(52) U.S. Cl. 424/439; 426/603; 426/583; 426/598; 426/580; 426/655

(57) ABSTRACT

A food product, being a spread comprising from 10-85 wt % of fat or a dairy based drink comprising from 0.05 to 1 wt % of kaempferol can advantageously be used to control blood pressure. Specifically kaempferol may be used for vasorelaxation.

### FIELD OF THE INVENTION

The invention relates to a composition comprising blood pressure lowering polyphenols, in particular vasoactive polyphenols.

### BACKGROUND OF THE INVENTION

Hypertension or high blood pressure is considered to be one of the main risk factors for Cardio Vascular Diseases (CVD). Compounds which have a blood pressure lowering effect are believed to achieve in a reduction of risk of CVD.

One of the mechanisms which regulates blood pressure is the renin-angiotensin system. This is a cascade of reactions leading to the formation of angiotensin II, which has a strong vasoconstrictive and hence blood pressure increasing effect.

ACE-inhibitors in food products are well known. Such food products have for instance been prepared by fermentation of milk or milk products (Hata, Y et al. (1996), American Journal of Clinical Nutrition 64, 767-771).

WO2005/068485 discloses the use of specific flavonoid compounds isolated from sedum sarmentosu bunge for preventing and treating hypertension. It relates to pharmaceutical compositions and food comprising flavonoid. The glycosylated form of kaempferol and is indicated for the prevention or treatment of hypertension via the mechanism of ACE inhibition. It does not disclose the use of the aglycon form of kaempferol; neither does it disclose the use of kaempferol as a vasorelaxing agent.

US 2004/132816 relates to a method for treating and/or preventing cardiovascular disease induced by hyperlipidemia. For this purpose the use of 0.1-10 wt % of kaempferol in the food or beverage is suggested. There is no relation of kaempferol with either vasorelaxation or hypertension mentioned.

US 2006/222682 discloses the incorporation of portions from the Moringa plant into neutraceutical beverage to reduce blood pressure. Kaempferol is mentioned as a suitable ingredient, however no specific amount is mentioned.

U.S. Pat. No. 6,187,314 discloses compounds from the Ginkgo Biloba leaves. It mentions the glycoside form of kaempferol. However nowhere is any effect on blood pressure let alone on vasorelaxation shown, nor is any specific amount of kaempferol disclosed.

WO 01/49285 discloses a capsule comprising 30 mg kaempferol per 100 g (=0.03 wt %). No spread or dairy based drink containing kaempferol is mentioned.

WO 02/14464 describes that increased intake of flavonols and flavanols reduces death by cardiac infarcts. There is no disclosure of kaempferol. Specific mentioning of neither decreasing hypertension nor vasorelaxation can be found.

WO 01/03687 discloses a method of inhibiting biosynthesis or bioactivity of endogenous steroid sex hormones by administration of a combination of phytosterols and phytoestrogens. Kaempferol is mentioned as a possible phytoestrogen compound. It is said that the by using the method woman can continue to enjoy the benefits of improved cardiovascular and skeletal health. No specific of the vasorelaxating properties of kaempferol are mentioned. It has also been suggested to use wine polyphenols for the prevention of cardiovascular diseases, for example EP 930 831 suggests the use of plant-derived flavanol compositions for example to inhibit oxidation of plasma LDL. However the addition of wine polyphenols to foods has various disadvantages. For example these polyphenols often lead to undesirable taste and color of the food products. Furthermore the addition of polyphenol-rich extracts, for example derived from wine or chocolate, to food product has the disadvantage that such extract comprises a mixture of multiple polyphenolic ingredients some of which may provide the desired functionality, while a great part of the polyphenol-rich extract is composed of non-functional or otherwise undesired compounds.

It is an object of the invention to formulate food products which comprise one or more polyphenolic compounds, whereby the type of polyphenolic compound and its amount are chosen such that on the one hand the vasoactive functionality, especially the ability to cause vasorelaxation is optimized, while on the other hand the amount of non-functional or otherwise undesired polyphenols can be

minimized, the amount of expensive polyphenol can be relatively low and undesired properties such as undesired taste and color of the product can be minimized.

Surprisingly it has been found that specific polyphenolic compounds when used in specific amounts in specific food products lead to vasorelaxation and therefore can have a positive contribution to the prevention or treatment of high blood pressure. When used in food products in specific amounts these food products generally have an acceptable taste and colour and can advantageously be used in a diet to promote a lowering of blood pressure, especially vasorelaxation.

### SUMMARY OF THE INVENTION

In a first aspect the invention relates to a fat based spread comprising from 10-85 wt % of fat, preferably 10-80 wt % vegetable fat, optionally in combination with up to 5 wt % of animal fat or marine oil and 10-90 wt % of water, wherein the spread comprises 0.05 to 1.0 wt % of kaempferol.

In a second aspect the invention relates to a drink, especially a dairy based drink, wherein the drink comprises from 10 to 99 wt % of a liquid protein base, for example a dairy base such as cow milk or yoghurt or a vegetable protein base such as soy milk, and 0.05 to 1.0 wt % of kaempferol.

Preferably the level of kaempferol in the food product is from 0.1 to 0.9 wt %, more preferred from 0.25 to 0.8 wt %, most preferred 0.4 to 0.75wt %, whereby the food product preferably is selected from the group of spreads and drinks.

### DETAILED DESCRIPTION OF THE INVENTION

Kaempferol (3,5,7-trihydroxy-2-(4-hydroxyphenyl)-4H-1-benzopyran-4-one) is a well-known polyphenolic compound which is commercially available in purified form e.g. 95% purity. Kaempferol is also present as a component in natural extracts such as for example wine extract. For the purpose of the invention kaempferol can be incorporated into the food product in any suitable form, for example as a relatively pure ingredient or as part of a natural extract comprising the kaempferol. For the purpose of the invention the amount of desired level of kaempferol can be achieved by any suitable method, for example the addition of suitable amounts of compositions comprising the kaempferol e.g. in purified form or as part of a natural extract.

Especially suitable natural extracts can for example be derived from grape juice, red wine, leek, broccoli, beans, Brussels sprouts, strawberries, witch-hazel, Delphinium and tea. Especially preferred sources of kaempferol are extracts from red wine, grape juice, broccoli, and black or green tea. The amount of such extracts in food products can be tailored depending on the concentration of kaempferol in the extract. Generally the concentration of such extracts in food products of the invention will be below 5 wt %, such as below 2 wt % or even below 2 wt %, examples of suitable levels of such extracts in food products according to the invention are 0.5 wt % or 0.75wt %. Kaempferol in plant extracts is usually in the glycoside form. The glycoside kaempferol is yellow colored and gives the flowers of Acacia decurrens and Acacia longifolia their color.

Another suitable form of kaempferol is the non-glycosilated form, or aglycon form.

Food products according to the invention are defined as products suitable for human consumption.

The food products according to the invention may be of any food type. They may comprise common food ingredients in addition to the food product, such as flavour, sugar, sweeteners, fruits, minerals, vitamins, stabilisers, thickeners, etc. in appropriate amounts.

Preferably, the food product comprises in addition to kaempferol 0.05-5.0 wt % K⁺. This cation has a beneficial effect of further lowering blood pressure when incorporated in the food products according to the invention.

Preferably, the food product also comprises one or more phytosterols, phytostanols and/or analogues or derivatives

thereof, especially the esterified derivatives thereof.

Typically, the phytosterols, phytostanols and their analogues and derivatives may be selected from one or more of phytosterols, phytostanols, synthetic analogues of

phytosterols and phytostanols and esterified derivatives of any of the foregoing, and mixtures of any of these. The total amount of such substances in a food product or food supplement is preferably from 0.01% to 20%, more preferably from 0.1% to 15%, still more preferably from 0.2% to 8%, and most preferably from 0.3% to 8% by weight of the food product composition.

Preferably the phytosterol or phytostanol is selected from the group comprising fatty acid ester of β-sitosterol, β-sitostanol, campesterol, campestanol, stigmasterol, stigmastanol and mixtures thereof.

The optional phytosterol or phytostanol materials recited above may optionally be provided in the form of one or more fatty acid esters thereof. Mixtures of esterified and non-esterified materials may also be used.

Preferably the food products according to the invention are spreads or drinks, more preferably fruit juice products or dairy drinks optionally with added fruit juice, dairy type products, frozen confectionary products or spreads/margarines. These preferred types of food products are described in some detail below and in the examples.

Fruit Juice Products

Examples of fruit juice products according to the invention are juices derived from citrus fruit like orange and grapefruit, tropical fruits, banana, peach, peer, strawberry, to which kaempferol and optionally one or more heart health ingredients are added. Fruit juice products may advantageously comprise a liquid protein base such a soy milk, cow milk or yoghurt, whereby typically the amount of fruit juice can be from 1 to 99 wt %, advantageously from 2 to 15 wt %.

Dairy Type Products

Examples of dairy products according to the invention are milk, dairy spreads, cream cheese, milk type drinks and yoghurt, to which kaempferol and optionally one or more further heart health ingredients are added. For the purpose of the invention soy milk based drinks are also considered as dairy products according to the invention, although for some applications the use of animal derived dairy bases such as cow milk or cow milk derived yoghurt is preferred.

The food product may be used as such as a milk or yoghurt type drink. Alternatively flavour or other additives may be added. A dairy type product may also be made by adding kaempferol to water or to a dairy product.

An example of a composition for a yoghurt type product is about 50-80 wt.% water, 0.1-1 wt.% kaempferol and optionally one or more heart health ingredients, 0-15 wt.% whey powder, 0-15 wt.% sugar (e.g. sucrose), 0.01-1 wt.% yoghurt culture, 0-20 wt.% fruit, 0.05-5 wt.% vitamins and minerals, 0-2 wt.% flavour, 0-5 wt.% stabilizer (thickener or gelling agent). To the yoghurt, fruit may be added.

A typical serving size for a yoghurt type product could be from 50 to 250 g, generally from 80 to 200 g.

Frozen Confectionery Products

For the purpose of the invention the term frozen confectionery product includes milk containing frozen confections such as ice-cream, frozen yoghurt, sherbet, sorbet, ice milk and frozen custard, water-ices, granitas and frozen fruit purees.

Preferably the level of solids in the frozen confection (e.g. sugar, fat, flavouring etc) is more than 3 wt.%, more preferred from 10 to 70 wt.%, for example 40 to 70 wt.%.

Ice cream will typically comprise 0 to 20 wt.% of fat, 0.1 to 1.0 wt.% kaempferol and optionally one or more heart health ingredients, sweeteners, 0 to 10 wt.% of non-fat milk components and optional components such as emulsifiers, stabilisers, preservatives, flavouring ingredients, vitamins, minerals, etc, the balance being water. Typically ice cream will be aerated e.g. to an overrun of 20 to 400%, more specific 40 to 200% and frozen to a temperature of from −2 to −200° C., more specific −10 to −30° C. Ice cream normally comprises calcium at a level of about 0.1 wt %.

Spreads

Advantageously the food product is an oil and water containing emulsion, for instance a margarine type spread. Oil and water emulsion is herein defined as an emulsion comprising oil and water and includes oil in water (O/W) emulsions and water in oil emulsions (W/O) and more complex emulsions for instance water-in-oil-in-water (W/O/W/O/W) emulsions. Oil is herein defined as including fat. Preferably the food product is a spread, frozen confection, or sauce. Preferably a spread according to the invention comprises 20-80 wt. % vegetable oil. Advantageously a spread has a pH of 4.2-6.0.

Spreads of the invention may comprise other ingredients commonly used for spreads, such as flavouring ingredients, thickeners, gellation agents, colouring agents, vitamins, emulsifiers, pH regulators, stabilizers etc. Common amounts of such ingredients as well as suitable ways to prepare margarines or spreads are well-known to the skilled person.

The invention will now be illustrated by means of the following examples.

### Example I

Material and Methods

Reactivity of Isolated Arteries

Segments of 2nd order mesenteric artery side branches were isolated from 14 weeks old male Spontaneously Hypertensive Rats (SHR). Two stainless-steel wires (diameter 40 μm) were inserted in the lumen of the arterial segments, which were then mounted in organ chambers between an isometric force transducer and a displacement device (Danish Myotechnology by J. P. Trading, Denmark). The organ chambers were filled with Krebs-Ringer bicarbonate solution which was maintained at 37° C. and continuously aerated with 95% O₂ and 5% CO₂. Before the actual experiments started, arterial segments were stretched to their individual optimal lumen diameter for mechanical performance, i.e. the diameter at which maximal contractile responses to noradrenaline (10 μmol/L) were obtained. In each experiment four second order mesenteric resistance arterial segments from one animal were mounted in individual organ chambers and studied in parallel. At the start of the experiments, all four arterial preparations were incubated during 20 min with capsaicin (1 μmol/L) to persistently desensitize sensory-motor nerves and to obtain a stable and considerable contractile response to potassium (K⁺, 40 mmol/L). During all the experiments superoxide dismutase (SOD, 5 U/ml) was present, to preserve stability of the metabolites.

Ingredients, Mixtures and Experimental Design

We tested the effects of the 35 different phenolic compounds using a Plackett-Burman screening design. The experiment consisted of 5 separate saturated 8 run Plackett-burman designs. Each design contained a subset of 7 phenolic compounds (Factors):

Design 1: Factors 1-7

Design 2: Factors 8-14

Design 3: factors 15-21

Design 4: Factors 22-28

Design 5: Factors 29-35.

One of the compounds tested was kaempferol (in this case compound 24). In each experiment we investigated whether a mixture of ingredients:
had a dilator effect during contraction induced by 40 mmol/L K+ (mixture concentration 0.1-100 umol/L)
modified contraction in response to 40 mmol/L K+ (100 umol/L mixture during 30 and 90 min)
modified endothelium-dependent vasodilatation in response to 0.001-10 umol/L acetylcholine (100 umol/L mixture), and
altered the bioavailability of NO, by performing concentration-response curves with the NO donor Na-nitroprusside (SNP, 0.0001-10 umol/L) during contraction induced by 40 mmol/L K+ (mixture concentration 100 umol/L).

The subsequent experimental steps are summarised in Table 1 Mixture concentrations refer to the concentrations of the individual ingredients in the mixtures. The volume of DMSO in the control bath was equal to the highest volume of DMSO, in which the mixes were dissolved, in the experimental baths. During registration of potential relaxing effects, the concentration of DMSO ranged from 0.3-1.2% for most mixtures (3 ingredients) and from 0.4 −-1.6% for the complex mixtures (6 ingredients).

For the analysis of the acute effects of the mixtures on K+-induced contraction, increasing concentrations of the mixtures (0.1-100 umol/L) were administered on top of the stable contractions and were left in contact with the arterial segments for 5-7 min to make sure that either no effect or a stable effect was reached. For the analyses of effects on the relaxing responses to Ach and SNP, the arterial segments were exposed for 30 min to a high concentration of the mixtures (100 umol/L), were then made to contract with K+and subsequently exposed to increasing concentrations of the vasodilator drugs. Between the Ach and SNP experiments, the tissues were maintained in the continuous presence of 100 umol/L of the mixture. In this way, the effects of different exposure times to the mixtures (7, 30 and 90 min) on K+-induced contraction, could be evaluated.

TABLE 1
Overview of the experimental design used to study
the effects of solvent (DMSO) and mixture of phenolic
compounds on the contractile responses, endothelium-
dependant vasodilation and dilator responses to exogenous
NO in isolated mesenteric resistance arteries of SHR.

| Segment | 1 | 2 | 3 | 4 |
|---|---|---|---|---|
| Pre-incubation | SOD (5 U/ml) | SOD (5 U/ml) | SOD (5 U/ml) | SOD (5 U/ml) |
| (30 min) | | | | |
| Contractile response | Potassium (K⁺) | Potassium (K⁺) | Potassium (K⁺) | Potassium (K⁺) |
| | (40 mM) | (40 mM) | (40 mM) | (40 mM) |
| Dilator response | DMSO | MIX A | MIX B | MIX C |
| | | (0.1-100 μM) | (0.1-100 μM) | (0.1-100 μM) |
| | wash | wash | wash | wash |
| Pre-incubation | SOD | SOD + MIX A | SOD + MIX B | SOD + MIX C |
| (30 min) | DMSO | (100 μM) | (100 μM) | (100 μM) |
| Contractile | K⁺ (40 mM) | K⁺ (40 mM) | K⁺ (40 mM) | K⁺ (40 mM) |
| response | | | | |
| Dilator response | Acetylcholine | Acetylcholine | Acetylcholine | Acetylcholine |
| | (ACH) | (ACH) | (ACH) | (ACH) |
| | (10⁻⁹-10⁻⁵ M) | (10⁻⁹-10⁻⁵ M) | (10⁻⁹-10⁻⁵ M) | (10⁻⁹-10⁻⁵ M) |
| | wash | wash | wash | Wash |
| Pre-incubation | SOD | SOD + MIX A | SOD + MIX B | SOD + MIX C |
| (30 min) | DMSO | (100 μM) | (100 μM) | (100 μM) |
| Contractile | K⁺ (40 mM) | K⁺ (40 mM) | K⁺ (40 mM) | K⁺ (40 mM) |
| response | | | | |
| Dilator response | Sodium | Sodium | Sodium | Sodium |
| | nitroprusside | nitroprusside | nitroprusside | nitroprusside |
| | (SNP) | (SNP) | (SNP) | (SNP) |
| | (10⁻¹⁰-10⁻⁵ M) | (10⁻¹⁰-10⁻⁵ M) | (10⁻¹⁰-10⁻⁵ M) | (10⁻¹⁰-10⁻⁵ M) |
| | wash | wash | wash | Wash |

Descriptive Statistics.

The following consecutive calculations were undertaken. During registration, isometric force (F) was converted into wall tension (WT =F/21, with 1 being the arterial segment length).

Active wall tension (AWT) was calculated by subtracting the resting wall tension.

All contractile responses (AWT in the presence of stimuli, solvent and mixes of ingredients) were next expressed as a percentage of the contractile response (AWT) to 10 uM noradrenaline at the end of the normalisation period, i.e. before exposure of the arterial segments to solvent or mixes of ingredients.

Effects of increasing concentrations of the solvent and of the mixes (0.1 to 100 umol/L) during contractile responses to 40 mmol/L K+, were calculated as % change of the level of pre-contraction. Next the effects of the mixes were corrected for the combined effects of time and solvent.

To calculate the effects of 30 min exposure to 100 umol/L of the mixes on the contractile response to 40 mmol/L K+ we took into account:
the response before exposure to the mix (a)
the response in the presence of the mix (b)
the response before exposure of a parallel control tissue, to the solvent (a′), and
the response during exposure of a parallel control tissue, to the solvent (b′).

With these we calculated the % change using the formula:

(((b−a·(b′/a′))/(a·(b′/a′)))·100.

For the solvent data, we used the mean of the observations in the solvent control experiments.

Responses to acetylcholine and Na-nitroprusside were analysed in terms of sensitivity (pD2=−log (M) EC50) and maximal effect (Emax) by least square sigmoidal curve fitting of individual concentration-response curves (Graphpad Prism 1.00, San Diego, Calif., USA). Findings in the presence of the mixtures of ingredients were subtracted from the findings in the presence of the respective concentrations of solvent.

The effects of acetylcholine were clearly biphasic, consisting of relaxations followed at higher concentrations by a reversal of the relaxations. Therefore, the analysis of sensitivity was limited to the relaxing component and two Emax were defined; one representing the maximal relaxation, the other one representing the response at the highest doses (3-10 umol/L).

Results

A total of 51 arteries of 13 SHR rats were used in this study. Their diameter ranged between 250 and 350 μm. At optimal diameter, the maximal contractile response of the arteries to noradrenaline averaged 4.65±0.17 N/m. We evaluated the effects of mixtures of compounds on contractile responses to 40 mmol/L K+, and on relaxation of K+-induced contraction by acetylcholine and Na-Nitroprusside. The contractile response to K+ averaged 72.01±2.36% of the maximal response to noradrenaline.

The solvent that was used in this study (DMSO) elicited a concentration-dependent relaxing effect during K+-induced contraction. Furthermore, prolonged exposure to the solvent resulted in progressive impairment of the contractile responses to 40 mmol/L K+. All observations with mixtures of ingredients were corrected for the solvent effects.

Several mixtures of compounds were tested in this way and by comparing the effect of different mixtures it could be shown that kaempferol at a low concentration had a significant influence on the dilation of the arteries, whereby the EC50 is obtained at a concentration of 3.0 micromolar of kaempferol. The maximum dilatation was obtained at 100 micromolar of kaempferol.

### Example II

Formulation of Food Products

Food products can be formulated which contain amounts of kaempferol sufficient to achieve a plasma concentration which is such that a positive influence on the dilation of the arteries can be expected, while avoiding unnecessary overdosing. To achieve this it is suggested that the preferred amount of kaempferol is from 0.05 to 1.0 wt % of the food product. This means that a typical serving size (say 10 to 200 g, for example 75 to 150 g for a drink and 10 to 30 g for a spread) can lead to plasma levels for kaempferol in the same order of magnitude as the concentrations as tested in example I, which was shown to have a positive effect on the dilation of the arteries.

Examples of Suitable Food Products are:

Spread

A commercially available margarine (Flora UK) is kept at 10 C, and subsequently 100 g of the margarine is mixed with 500 milligrammes of kaempferol (calculated as % purity)to obtain a kaempferol containing spread which when used, for example at a dose of 20 grammes per day, can advantageously be used by consumers who are interested to control their blood pressure.

A commercially available yoghurt based drink containing 3 wt phytosterol ester and sold in containers of 100 ml (Pro-activ UK) is kept at 10 C and subsequently 500 mg of kaempferol (calculated as 100% purity) is mixed into the content of one bottle to obtain a drink which, when used, for example at a dose of 75 to 150 ml per day, can advantageously be used by consumers who are interested to control their blood pressure.

1. A fat based spread comprising from 10-85 wt % of fat and 10-90 wt % of water, wherein the spread comprises 0.05 to 1.0 wt % of kaempferol, more preferred 0.1 to 0.9 wt % of kaempferol, most preferred 0.4 to 0.75 wt % of kaempferol.

2. A drink, especially a dairy based drink, wherein the drink comprises from 10 to 95 wt % of a dairy base such as cow milk, soy milk or yoghurt, especially preferable cow milk or yoghurt, and 0.05 to 1.0 wt % of kaempferol, more preferred 0.1 to 0.9 wt % of kaempferol, most preferred 0.4 to 0.75 wt % of kaempferol.

3. A fat based spread according to claim 1 comprising from 20-85 wt % of vegetable fat and optionally 0-5 wt %, for example from 0.1 to 2 wt % of animal fat or marine oil.

4. A fat based spread according to claim 1 comprising from 0.1 to 15 wt % p, more preferred from 0.3 to 8 wt % of phytosterols, phytostanols or derivatives thereof, preferably fatty acid ester derivatives thereof.

5. A spread according to claim 1, comprising from 0.05 to 5.0 wt % Potassium ions per kg.

6. A spread according to claim 1 wherein the kaempferol is in the aglycon form.

7. Food product comprising 0.05 to 1.0 wt % of kaempferol for use in vasorelaxation.

8. Food product according to claim 7 wherein the food product is a fat based spread comprising from 10-85 wt % of fat 0.05, or a drink comprising a dairy base from 10 to 95 wt % for use in vaso-relaxation.

9. Food product according to claim 7 wherein the kaempferol is in the form of a plant extract.

10. Food product according to claim 7 wherein the kaempferol is in the aglycon form.

11. Use of 0.05 to 1.0 wt % of kaempferol in the preparation of a food product for use in vaso-relaxation.

12. Use according to claim 11 wherein the kaempferol is in the aglycon form or in the glycosilated form.

13. A drink according to claim 2 comprising from 0.1 to 15 wt % p, more preferred from 0.3 to 8 wt % of phytosterols, phytostanols or derivatives thereof, preferably fatty acid ester derivatives thereof.

14. A drink according to claim 2, comprising from 0.05 to 5.0 wt % Potassium ions per kg.

15. A drink according to claim 2 wherein the kaempferol is in the aglycon form.
